(19) United States
(12) Patent Application Publication
(10) Pub. No.: US 2026/0157817 A1
(43) Pub. Date: Jun. 11, 2026

(54) SURGICAL PACKAGES FOR OPHTHALMIC SURGERY SETUP ROBOT

(71) Applicant: Alcon Inc., Fribourg (CH)

(72) Inventors: Patrick TERRY, Goleta, CA (US); Satish YALAMANCHILI, Irvine, CA (US); Nanhong LOU, Southlake, TX (US)

(21) Appl. No.: 19/407,060

(22) Filed: Dec. 3, 2025

Related U.S. Application Data
(60) Provisional application No. 63/730,252, filed on Dec. 10, 2024.

(51) Int. Cl. A61B 50/30; A61B 34/30; A61B 90/94; A61B 90/98; B25J 9/16; B25J 13/08; B65B 69/00; B65D 75/58

(52) U.S. Cl. CPC A61B 50/30; A61B 34/30; A61B 90/94; A61B 90/98; B25J 9/1694; B25J 13/089; B65B 69/00; B65D 75/5855

(57) ABSTRACT

In certain embodiments, a surgical package is described that facilitates opening by an end effector of a robotic system. The surgical package includes a body defining an interior volume configured to store one or more surgical items therein. A first portion of the body defines a first opening into the interior volume. The surgical package further includes a cover defining a sealing surface configured to contact the first portion to cover the first opening and isolate the interior volume from ambient, and a projecting interface that extends beyond an areal extent of the first portion. The projecting interface is configured to be manipulated by the end effector to overcome a sealing force between the cover and the first portion.

### CROSS-REFERENCE TO RELATED APPLICATIONS

This application claims the benefit of U.S. Provisional Patent Application No. 63/730,252, filed Dec. 10, 2024, which is incorporated by reference herein in its entirety, and is hereby expressly made a part of this specification.

### INTRODUCTION

The present disclosure relates to ophthalmic surgery.

Common ophthalmic surgical treatments include cataract surgery, glaucoma treatments, retinal membrane peeling, vitrectomy, and retinal reattachment. The structures of the eye are extremely small and delicate. Ophthalmic surgery is therefore extremely sophisticated and becoming an ophthalmic surgeon requires many years of training. The time of an ophthalmic surgeon spent in the operating room is therefore a very valuable resource.

### SUMMARY

In certain embodiments, a surgical package is described that facilitates opening by an end effector of a robotic system. The surgical package includes a body defining an interior volume configured to store one or more surgical items therein. A first portion of the body defines a first opening into the interior volume. The surgical package further includes a cover defining a sealing surface configured to contact the first portion to cover the first opening and isolate the interior volume from ambient, and a projecting interface that extends beyond an areal extent of the first portion. The projecting interface is configured to be manipulated by the end effector to overcome a sealing force between the cover and the first portion.

In certain embodiments, a system includes a robotic arm comprising at least one end effector, and a controller configured to control the robotic arm to position the end effector relative to a surgical package comprising a body and a cover defining a projecting interface. The projecting interface extends beyond an areal extent of a first portion of the body that defines a first opening into an interior volume of the body. The controller is further configured to control the end effector to manipulate the projecting interface to overcome a sealing force between the cover and the first portion.

In certain embodiments, a method is described for operating an end effector of a robotic system to open a surgical package. The method includes controlling a robotic arm to position the end effector relative to the surgical package. The surgical package includes a body, and a cover defining a projecting interface. The projecting interface extends beyond an areal extent of a first portion of the body that defines a first opening into an interior volume of the body. The method further includes controlling the end effector to manipulate the projecting interface to overcome a sealing force between the cover and the first portion.

### BRIEF DESCRIPTION OF THE DRAWINGS

FIG. 1A is a schematic diagram of an operating environment including a setup robot, according to one or more embodiments.

FIG. 1B illustrates an exemplary setup robot including a robotic arm mounted to a guide rail, according to one or more embodiments.

FIG. 1C illustrates another exemplary setup robot including two robotic arms mounted to a guide rail, according to one or more embodiments.

FIG. 1D illustrates a tray holding supplies for an ophthalmic treatment, according to one or more embodiments.

FIG. 1E illustrates components for operating a setup robot, according to one or more embodiments.

FIG. 2 illustrates an exemplary surgical package to facilitate opening by an end effector of a robotic system, according to one or more embodiments.

FIG. 3 is a diagram illustrating an exemplary end effector for manipulating a surgical package, according to one or more embodiments.

FIG. 4A is a diagram illustrating an exemplary surgical package having a film extending across an opening extending through a body, according to one or more embodiments.

FIG. 4B is a diagram illustrating exemplary end effector manipulation of a surgical package having a film extending across an opening extending through a body, according to one or more embodiments.

FIG. 5A is a diagram illustrating an exemplary surgical package having a tape loop attached to a surface of a projecting interface, according to one or more embodiments.

FIG. 5B is a diagram illustrating exemplary end effector manipulation of a surgical package having a tape loop attached to a surface of a projecting interface, according to one or more embodiments.

FIG. 6A is a diagram illustrating an exemplary surgical package having opposing tabs for manipulation by an end effector, according to one or more embodiments.

FIG. 6B is a diagram illustrating exemplary end effector manipulation of a surgical package having opposing tabs, according to one or more embodiments.

FIG. 7A is a diagram illustrating exemplary end effector manipulation of a surgical package with portions of a cover having an attachment force, according to one or more embodiments.

FIG. 7B is a diagram illustrating exemplary end effector manipulation of a surgical package with portions of a cover having an attachment force, according to one or more embodiments.

FIG. 8 is a diagram illustrating an exemplary surgical package having a retention mechanism, according to one or more embodiments.

FIG. 9 is an exemplary method of operating an end effector of a robotic system to open a surgical package, according to one or more embodiments.

FIG. 10A is a diagram illustrating an exemplary surgical package with a projecting interface having an identifier comprising one or more visual attributes, according to one or more embodiments.

FIG. 10B is a diagram illustrating an exemplary surgical package with a projecting interface having an identifier comprising a machine-readable code, according to one or more embodiments.

FIG. 10C is a diagram illustrating an exemplary surgical package with a projecting interface having an identifier comprising a Radio Frequency Identification (RFID) tag, according to one or more embodiments.

FIG. 10D is a diagram illustrating an exemplary surgical package with a projecting interface having an identifier comprising a machine-readable code, according to one or more embodiments.

### DETAILED DESCRIPTION

In certain embodiments, a system for ophthalmic surgery may include one or more patient stations configured to facilitate the performance of ophthalmic procedures by the surgeon. Each patient station may include a patient support (such as a bed, chair, etc.), a surgical microscope mounted to an adjustable support, a surgical table for supporting a tray with items that will be used during the ophthalmic procedure, and a disposal station for disposing, storing or processing used items from the tray. A supply area (such as a table, bench, cabinet, etc.) stores the tray (or trays) that will be used during the ophthalmic procedure.

The system also includes cameras positioned in various locations of the operating environment to provide views of the patient stations and the supply area, a setup robot, and a controller that is coupled to the cameras and the setup robot. Generally, the setup robot includes a robotic arm with a camera and an end effector. The controller is configured to perform various functions related to the ophthalmic procedure, such as processing image data that are received from the cameras, controlling the setup robot before, during, and after the ophthalmic procedure, etc. For example, the controller may command the setup robot to prepare the patient station for the ophthalmic procedure by transferring one of the trays from the supply area to the surgical table. In other examples, the controller may command the setup robot to pass items to the surgeon from the tray on the surgical table, place items received from the surgeon back on the tray on the surgical table, discard items received from the surgeon into the disposal station, transfer the tray from the surgical table to the supply table after the ophthalmic procedure is completed, etc.

The items needed for the ophthalmic procedure may include instruments, consumable products, structures to be implanted, etc. These items arrive at the surgical facility in sealed packages to protect the sterility of each item prior to the ophthalmic procedure. Unfortunately, surgical staff must manually load each tray by opening each sealed package and placing the item on the tray. The required motions that surgical staff perform when identifying and opening these sealed packages are technically complex, and surgical staff training bottlenecks may prevent the optimum efficiency. Additionally, the inventory of the items placed on the trays is not automatically tracked, and may be dependent upon the diligence of the surgical staff. Because high-volume ophthalmic procedure facilities require a large number of surgical staff, staffing shortages for scrub-techs, nurses, etc., impact the efficiency of these facilities. Staffing shortages invariably lead to the need for surgical staff to work overtime hours, which causes stress and high mental strain.

Certain embodiments of the present disclosure advantageously replace the manual loading process for each tray with a robotic-based process that reduces surgical staffing requirements, does not require training of new surgical staff or re-training of current surgical staff, and can automatically track inventory. In certain embodiments, a system for ophthalmic surgery includes robotic arms with multiple end effectors that interact with surgical packages containing items used during ophthalmic surgery in order to identify the items, open the surgical packages, and release the items from the surgical package (such as onto the tray).

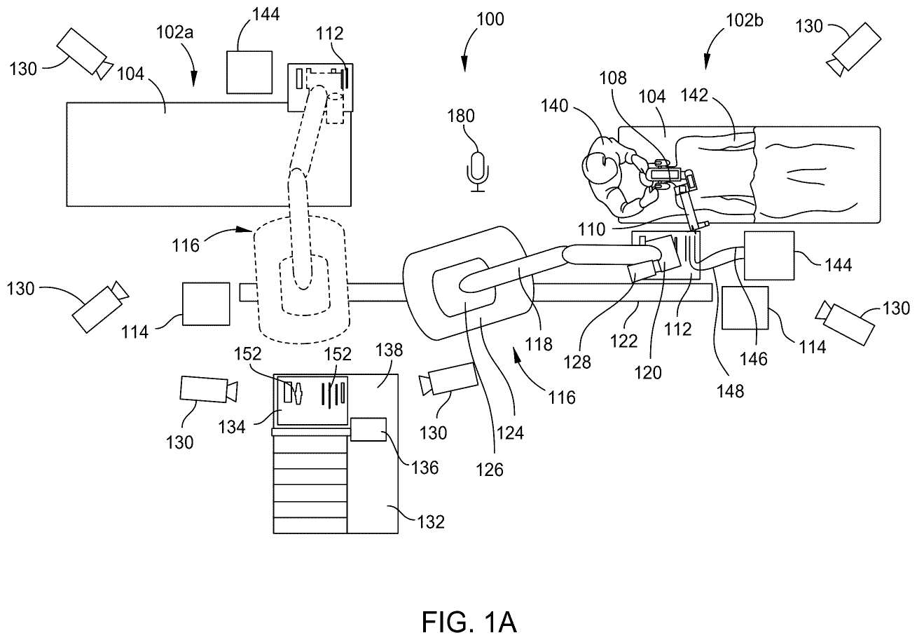

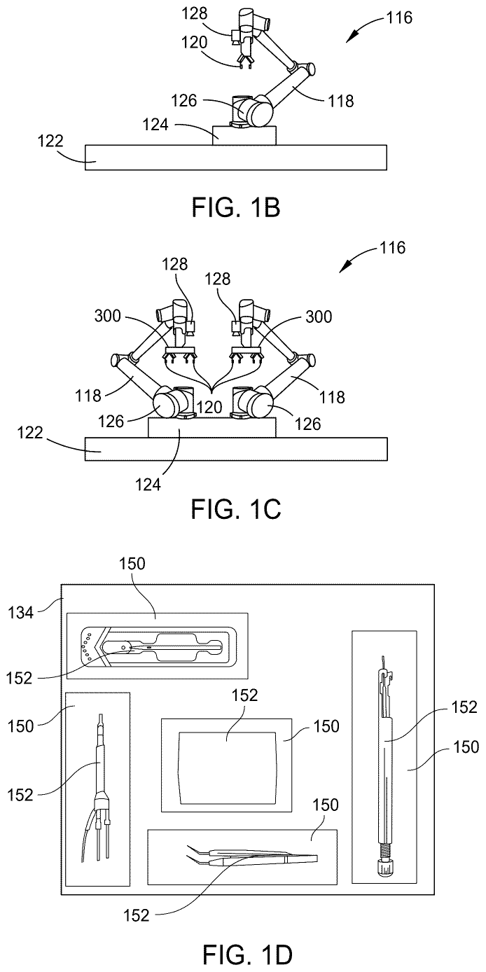

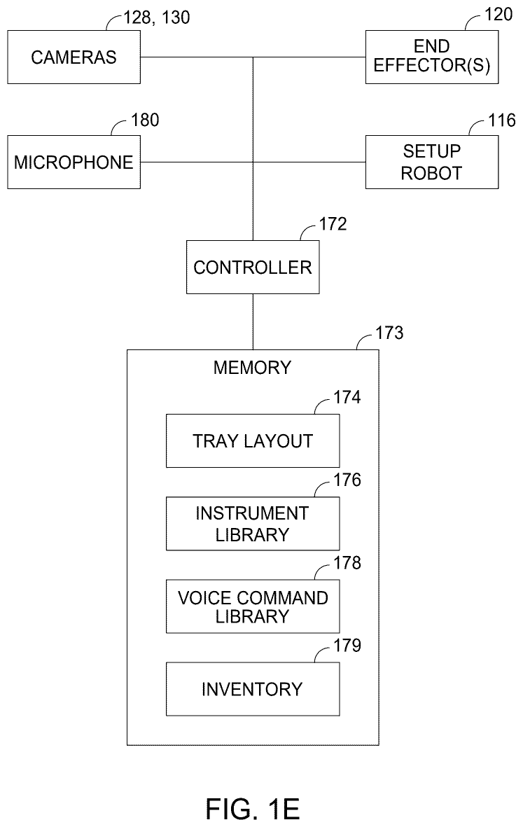

Referring to FIGS. 1A, 1B, 1C, an operating environment 100 includes two or more stations 102a, 102b, and each station 102a, 102b includes a patient support 104 for supporting a patient 142, such as a bed, chair, or other type of support. Although the system and methods disclosed herein are advantageously used with multiple stations 102a, 102b, a single station 102a may also be used.

Each station 102a, 102b may include other equipment, such as an ophthalmic microscope 108 mounted to an adjustable support 110. In some embodiments, a single ophthalmic microscope 108 may be used, and the adjustable support 110 facilitates movement of the ophthalmic microscope 108 between station 102a and station 102b.

Each station 102a, 102b may include a surgical table 112 and a disposal station 114. The surgical table 112 provides a surface for supporting surgical supplies, such as a tray containing the items needed during the ophthalmic procedure. The disposal station 114 may include a waste bin, an autoclave, a collection bin for items to be sanitized elsewhere, a hazardous material disposal bin, a receptacle for storing or processing used surgical supplies, or a combination of these containers.

One or more setup robots 116 may be positioned in the operating environment 100. In certain embodiments, a single setup robot 116 may be used, and the setup robot 116 may prepare one station 102a (102b) while the other station 102b (102a) is in use. FIG. 1A depicts a setup robot 116 in a first position supporting station 102a (solid line), and in a second position supporting station 102b (dotted line). In other embodiments, a setup robot 116 may be provided for each station 102a, 102b, and FIG. 1A depicts a setup robot 116 supporting station 102a (solid line), and another setup robot 116 supporting station 102b (dotted line).

In the embodiments depicted in FIGS. 1A, 1B, the setup robot 116 includes a robotic arm 118. The robotic arm 118 may be a serial robotic arm extending from a shoulder joint 126, and may have 4 to 8 (or more) degrees of freedom. The degrees of freedom may be sufficient to position the end effector 120 of the robotic arm 118 at various three-dimensional positions and orientations within the working envelope of the robotic arm 118. The degrees of freedom may include one (or more) degrees of freedom of the end effector 120, such as a gripper, a tool changer, etc.

In the embodiment depicted in FIG. 1B, the end effector 120 may be a gripper for holding and releasing objects (such as gripper 320 depicted in FIG. 3). The gripper may be any structure configured to hold, manipulate, and release objects. The gripper may be configured to hold, manipulate, and release objects that have structures or features specifically designed to cooperate with the gripper. The gripper may be a mechanical gripper (such as a parallel-jaw gripper, three-finger gripper, a needle gripper, etc.), an electromagnetic gripper, a pneumatic gripper, a hydraulic gripper, etc.

The setup robot 116 may include a rail actuator 124 that is mounted a rail 122 (such as a linear rail, etc.). The rail actuator 124 may include a motor and a gear, wheel, or other structure that engages the rail 122. The gear is driven by the motor to translate the setup robot 116 to different positions along the rail 122. The rail 122 may be mounted to a floor, ceiling, or wall of the operating environment 100. Multiple rails and corresponding actuators may be used to implement a two-or three-dimensional translating gantry. In other embodiments, the rail actuator 124 does not include a motor, and the setup robot 116 may be manually translated along the rail 122 and locked in position.

Precise positioning of the end effector 120 may be performed in various ways. In certain embodiments, a kinematic state of the setup robot 116 and a known mapping of objects in the operating environment 100 are used along with obstacle detectors to position the end effector 120. Cameras or other local positioning system (LPS) may not be needed. In certain other embodiments, one (or more) cameras 128 may mounted to the robotic arm 118 on or near the end effector 120. For example, a camera 128 may be mounted within 15 cm of, and rigidly coupled to, the end effector 120. Images from the camera 128 may be processed to determine the location and orientation of the end effector 120, and used as feedback to control the robotic arm 118. In certain other embodiments, several cameras 130 may be distributed around the operating environment 100 in order to view the end effector 120. Visual markers may be attached to the end effector 120 and the robotic arm 118 to facilitate image recognition in the image data from the cameras 130. Image data from the cameras 130 may then be processed to determine the position and orientation of the end effector 120. The position and orientation data are used to control the robotic arm 118 to achieve a desired position and orientation of the end effector 120.

A supply area 132, such as a table, bench, cabinet, etc., may be positioned in the operating environment 100 within the operating envelope of the setup robot 116. The supply area 132 may support one or more trays 134. Each tray 134 may be loaded with items needed during an ophthalmic procedure. In some scenarios, more than one tray 134 may be used for a single ophthalmic procedure. The supply area 132 may include a gate 136 that allows trays 134 to drop, slide, or otherwise move into a pickup area 138. For example, trays 134 may be arranged according to a schedule of ophthalmic procedures such that each tray 134 may be retrieved by the setup robot 116 for each ophthalmic procedure in the schedule. In another example, during an ophthalmic procedure on a patient 142 in station 102b, the setup robot 116 may retrieve a tray 134 for the next ophthalmic treatment in station 102a, and place the tray 134 onto the surgical table 112 of station 102a.

In some embodiments, each station 102a, 102b may include a console 144 that provides ports for fluid connections (e.g., tubes 146 for supplying vacuum pressure and/or an infusion fluid), electrical connections (e. g, electrical lines 148 for supplying power and/or signals), or other types of ports. The setup robot 116 may prepare each station 102a, 102b for surgery by connecting each tube 146 and electrical line 148 between the console 144 (or other housing for a port) and an instrument, such as an instrument in the tray 134.

In the embodiment depicted in FIG. 1C, the setup robot 116 includes a first robotic arm 118 and a second robotic arm 118. Each robotic arm 118 may be a serial robotic arm, and may have 4 to 8 (or more) degrees of freedom. Each robotic arm 118 may have a dual end effector arm 300. Each dual end effector arm 300 may include two end effector arms 310, 312 (see FIG. 3), and each end effector arm 310, 312 may have an end effector 120. Each end effector arm 310, 312 may have 1 to 4 (or more) degrees of freedom. The degrees of freedom of the robotic arms 118 and the end effector arms 310, 312 may be sufficient to position each end effector 120 at various three-dimensional positions and orientations within the working envelope of the respective robotic arm 118. The end effector 120 may be a gripper, a tool changer, etc. For example, the gripper may be a mechanical gripper (such as a parallel-jaw gripper, three-finger gripper, a needle gripper, etc.), an electromagnetic gripper, a pneumatic gripper, a hydraulic gripper, etc.

In the embodiment depicted in FIG. 1D, each tray 134 supports a number of items 152 in respective recesses 150. Each item 152 may be an instrument, a consumable product, a structure to be implanted, or other item for use during an ophthalmic treatment. In other embodiments, the tray 134 does not include recesses 150, and the items 152 simply rest on the surface of the tray 134.

There are many items 152 that may be used to perform any number of ophthalmic treatments such as phacoemulsification and IOL placement, vitrectomy, glaucoma surgery, retinal attachment, refractive surgery (laser-assisted in situ keratomileusis (LASIK), small incision lenticule extraction (SMILE), implantable contact lens (ICL), etc.), or other ophthalmic treatments. A non-limiting list of example items 152 includes a sideport incision instrument, topical or injected anesthesia and corresponding syringe or other dispenser, cystotome, balanced salt solution (BSS), centurion handpiece, silicone, metal, or polymer irrigation/aspiration (I/A) tip, ophthalmic viscosurgical device, tryphan blue and applicator, forceps, OVD removal tool, fluidics management system (FMS) pack, metal or plastic handpiece, pre-loaded disposable or reusable intraoptical lens (IOL) injector, primary incision instrument, irrigation/hydrodissection tool, sutures, drapes, etc.

In certain embodiments, the layout of the tray 134 is known such that the controller for the setup robot 116 does not require visual recognition of the item within each recess 150. Instead, the controller may simply position the end effector 120 (such as a gripper) at a known location of a recess 150 containing an item 152 and lift the item 152 from the tray. In certain embodiments, the items 152 and the recesses 150 may include markings, text, or other computer-readable symbols that may be used by the controller to identify the item 152 located in a particular recess 150.

In the embodiment depicted in FIG. 1E, the setup robot 116, the end effector(s) 120, the cameras 128, 130, and the microphone 180 may be coupled to a controller 172 using wired connections (such as USB, Ethernet, etc.) or wireless connections (such as Bluetooth, WiFi, etc.). The controller 172 may be coupled to a memory 173. The controller 172 may be a general-purpose computer, programmable logic controller (PLC), or other electronic device programmed to perform the functions ascribed herein to the controller 172. Memory 173 may store instructions to be executed by controller 172, data, and other information, such as a tray layout 174, an instrument library 176, a voice command library 178, an inventory 179, etc.

The controller 172 may access a tray layout 174 for each tray 134 to be used for each ophthalmic procedure. The tray layout 174 may include an identifier of each tray 134 enabling the tray 134 to be identified, such as a marking, text, or other symbol affixed to the tray 134. The tray layout 174 may include a specification of the location of recesses 150 and an identifier of the item 152 positioned within each recess 150.

In some embodiments, to further facilitate the identification of items 152, the controller 172 may access the instrument library 176. For each type of item 152, the instrument library 176 may include such information as a marking, text, or other symbol that uniquely identifies the item 152, a position of each item 152 in each tray layout 174, a three-dimensional model of the item 152 enabling the items 152 to be identified in images from the cameras 128, 130, one or more two-dimensional images from different angles, or other data to facilitate machine identification of each type of item 152.

The controller 172 may be configured to evaluate images from cameras 128, 130, determine that the tray 134 in the pickup area 138 is the correct tray for of a scheduled ophthalmic procedure by detecting identification data in the images, cause the setup robot 116 to grab the tray 134 and move the tray 134 to the surgical table 112 of a station 102a, 102b for which the ophthalmic treatment is scheduled. As shown in FIG. 1A, the range of motion of the setup robot 116 along the rail 122 enables the setup robot 116 to place a tray 134 on the surgical tables 112 of both stations 102a, 102b.

The controller 172 may be configured to identify used items 152 returned to the tray 134 in the images from cameras 128, 130, and move the used items 152 to a disposal station 114 to be disposed of or disinfected for subsequent use.

In certain embodiments, the controller 172 may be configured to grasp an item 152 from a tray 134 and pass the item 152 to the surgeon 140 in response to a voice command. For example, the controller 172 may access the voice command library 178, which includes voice commands that specify an action and an identifier of an item 152, such as an action to pass an item 152 from a tray 134 to a surgeon, an action to receive an item 152 from a surgeon and pass the item 152 to the disposal station 114, etc. The microphone 180 may be centrally-located in the operating environment 100 to detect the voice commands of the surgeon 140. In other embodiments, each station 102a, 102b may have a corresponding microphone 180.

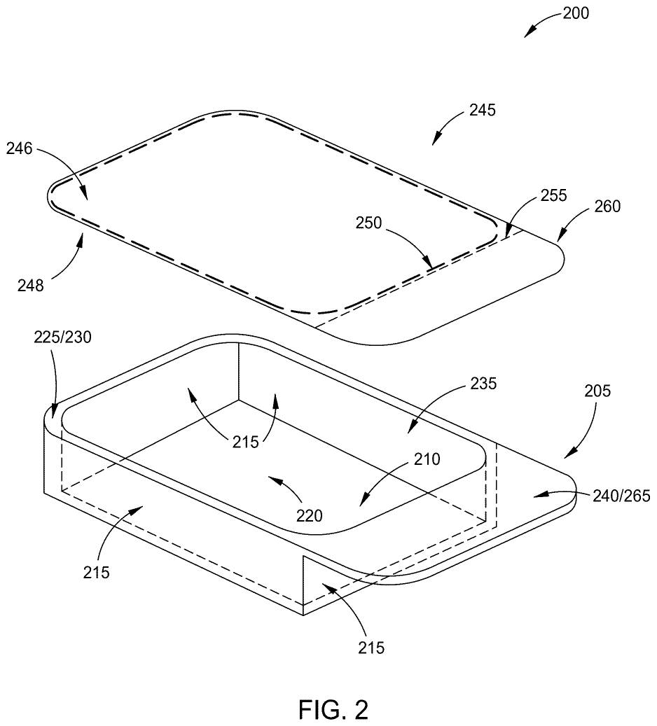

FIG. 2 illustrates an exemplary surgical package 200 to facilitate opening by an end effector of a robotic system, according to one or more embodiments. The features illustrated in FIG. 2 may be used in conjunction with other embodiments, e.g., the surgical package 200 may be manipulated by the setup robot 116 of FIG. 1.

The surgical package 200 comprises a body 205 and a cover 245. The body 205 comprises a base 210 and several sidewalls 215 that project from the base 210 (as shown, each sidewall 215 projects in a generally upward direction). The body 205 (e.g., the base 210 and sidewalls 215 collectively) defines an interior volume 220 of the surgical package 200. The interior volume 220 is dimensioned to store one or more surgical items therein (e.g., any of the various items 152 that are discussed above) and generally provide a sterile environment for storage. During operation of the setup robot 116, the surgical package 200 may be manipulated by the setup robot 116, and the one or more surgical items may be released from the surgical package 200 (e.g., onto the tray 134 of FIG. 1D) to be used by a surgeon or technician during an ophthalmic procedure.

The body 205 may be formed of any suitable material(s) that can be sterilized and that can maintain the sterility of the interior volume 220. The body 205 may include rigid materials and/or compliant materials. Some non-limiting examples of materials used in the body 205 include polypropylene (PP) and polyethylene (PE), as well as combinations thereof, which may be formed as one or more layers of spunbond non-woven fabric(S) and/or meltblown non-woven fabric (M) (e.g., S, SS, SMS, SMMS, SSMMS, SMMMS, and so forth). Other non-limiting examples of the materials include woven fabrics such as cotton and/or polyester, papers such as kraft paper and crepe paper, plastic films such as PE and polyethylene terephthalate (PET), foils, laminates, and so forth. In some embodiments, the material(s) composing the body 205 are reusable, e.g., material(s) that are capable of being sterilized multiple times, and having suitable strength to undergo multiple cycles of applying and removing the cover 245.

A first portion 225 of the body 205 is defined by the upper surfaces of the sidewalls 215. As shown, the upper surfaces together form a continuous rim 230 of the body 205 that has a rounded rectangular profile. Other sizes and shapes for the first portion 225 are also contemplated. The first portion 225 (here, the rim 230) defines an opening 235 into the interior volume 220, such that covering the opening 235 operates to isolate the interior volume 220 from ambient.

The cover 245 is disposed over the body 205 and has a rounded rectangular shape corresponding to the body 205 including the first portion 225. In some embodiments, the cover 245 has a thickness that is much less than its length and width. For example, the cover 245 may be implemented as a “peel tab” (e.g., a plastic tape or ribbon) having a nominal thickness. Other sizes and/or shapes of the cover 245 are also contemplated. For example, the cover 245 may be coextensive with the top surface of the body 205, may project past the extent of the body 205, or may extend beyond the opening 235 but not to the full extent of the body 205.

The cover 245 may be formed of any suitable material(s) that can be sterilized and that can maintain the sterility of the interior volume 220. The cover 245 may include rigid materials and/or compliant materials. Some non-limiting examples of the cover 245 include plastic films such as PE and polyethylene terephthalate (PET). In some embodiments, the material(s) composing the cover 245 are reusable, e.g., material(s) that are capable of being sterilized multiple times, and having suitable strength to undergo multiple cycles of being applied and removed from the body 205.

The cover 245 defines an upper surface 246 and an opposing sealing surface 248 that is contacted to the first portion 225 to cover the opening 235. As shown, the sealing surface 248 is substantially planar and corresponds to the profile of the rim 230 (e.g., the upper surfaces of the sidewalls 215). In other embodiments, the sealing surface 248 may have a different profile that corresponds to the profile of the rim 230. A portion of the sealing surface 248 that contacts the first portion 225 is shown as outline 250.

The cover 245 may removably attach to the body 205 using any suitable means that enable an end effector (e.g., the end effector 120) to manipulate the cover 245 and overcome a sealing force between the cover 245 and the first portion 225. In some embodiments, an adhesive layer may be arranged between, and provides the sealing force between, the first portion 225 and the sealing surface 248 (e.g., at the outline 250). The end effector 120 applies a grasping or gripping force to the cover 245 and gradually overcomes the sealing force provided by the adhesive layer (e.g., peeling the cover 245 back and away from the first portion 225. In other embodiments, the cover 245 may be formed of rigid material(s) and disposed in a channel (or on a rail) or other structure of the body 205 that provides the sealing force between, the first portion 225 and the sealing surface 248 (e.g., at the outline 250). The end effector 120 applies a grasping or gripping force along edges of the cover 245, sliding the cover 245 along the channel, rail, or other structure of the body 205 to overcome the sealing force. For example, pads may be formed on opposing sides of the cover 245, and the end effector 120 medially depresses the pads to release a retention mechanism and allow the cover 245 to slide.

The cover 245 further defines a projecting interface 260 that extends beyond an areal extent (represented by a dashed line 255 extending across the cover 245) of the first portion 225, which may be along one or more dimensions. The projecting interface 260 is generally movable relative to the portion of the cover 245 that contacts the first portion 225 of the body 205, e.g., deformable or deflectable. In some embodiments, the projecting interface 260 may be visually demarcated, e.g., a pivoting interface that is arranged at or near the dashed line 255. In some embodiments, the projecting interface 260 extends beyond the extent of the body 205 (e.g., overhangs the body 205) along one dimension, e.g., corresponding to a longitudinal axis of the body 205.

In some embodiments, and as shown, the body 205 further defines a tab 240 (also referred to as a second portion 265 of the body 205) that extends outward from an upper portion of a sidewall 215. In some embodiments, the tab 240 is integrally formed with the first portion 225. In some embodiments, the tab 240 is positioned such that a top surface of the tab 240 is coplanar with the rim 230.

The projecting interface 260 of the cover 245 may fully or partially overlap with the tab 240, or as mentioned above, may extend beyond the extent of the tab 240. In some embodiments, the projecting interface 260 may contact the top surface of the tab 240, but is not adhered or otherwise joined to the tab 240.

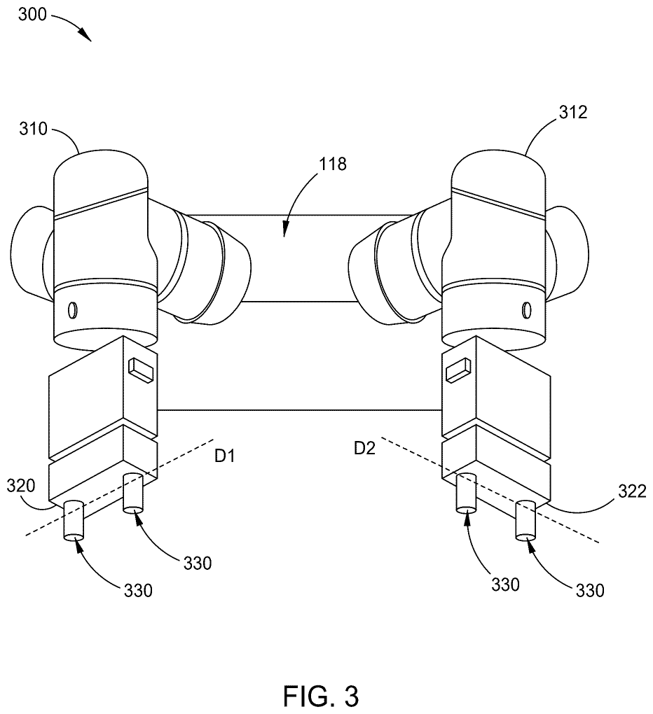

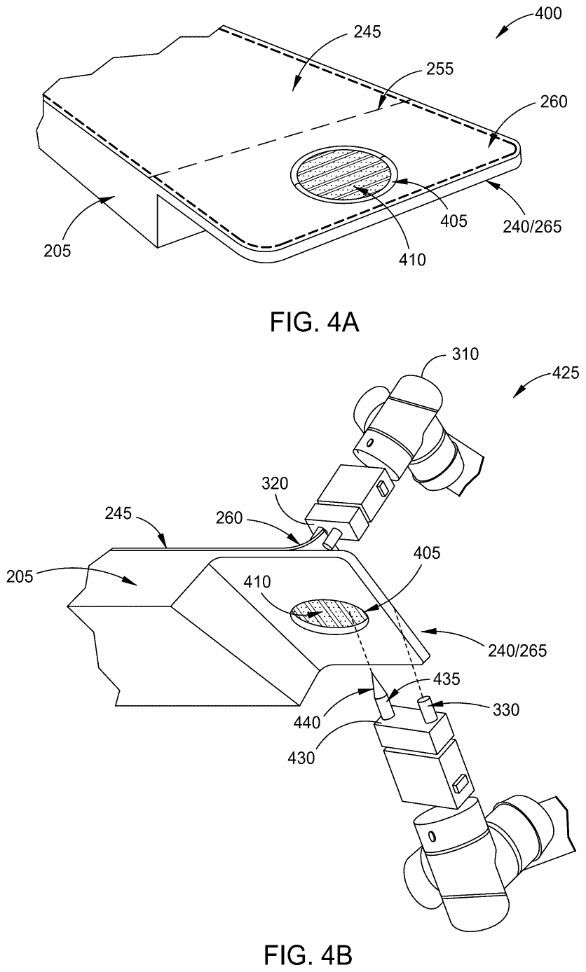

In the operating environment 100, manipulation of the surgical package 200, and specifically opening the surgical package 200 using the end effector(s) 120 to release items stored therein, poses a significant technical challenge. The various embodiments described herein, and discussed below with respect to FIGS. 4A-10B, provide a number of improvements to the surgical package 200 that enhance its ability to be manipulated by the end effector(s) 120, and therefore facilitate opening the surgical package 200 by the end effector(s) 120. For example, the projecting interface 260 may include features that enable the position and orientation of the surgical package 200 to be more easily identified within the operating environment 100, as well as features that allow the end effector(s) 120 to grasp and manipulate different portions of the surgical package 200 with greater precision.

In certain embodiments, a custom surgical package may comprise a set of surgical packages 200 with various items that are tailored for a specific ophthalmic procedure. While all of the items of the custom surgical package are removed from the surgical packages 200 and placed on the tray 134 before the ophthalmic procedure, not all of the items may be used during the ophthalmic procedure. Tracking the items from a custom surgical package that are actually used during the ophthalmic procedure allows the contents of the custom surgical package to be optimized to reduce waste.

In other words, embodiments of the present disclosure may advantageously provide fulfillment and supply chain business intelligence related to custom surgical packages and the ability to realize cost savings by being more precise about the contents of the custom surgical packages.

FIG. 3 is a diagram illustrating exemplary end effectors for manipulating a surgical package, according to one or more embodiments. The features illustrated in FIG. 3 may be used in conjunction with other embodiments, such as grasping and opening the surgical package 200 of FIG. 2.

In some embodiments, a dual end effector arm 300 includes an end effector arm 310 and an end effector arm 312. The dual end effector arm 300 is coupled to the end of the robotic arm 118 of the setup robot 116, such as the first robotic arm 118 (depicted in FIGS. 1B, 1C) or the second robotic arm 118 depicted in FIG. 1C.

The grippers 320, 322 represent example implementations of the end effectors 120 and are coupled to the end of the respective effector arms 310, 312. The grippers 320, 322 are configured to grip, hold, manipulate, and release the surgical package 200, and each is operatively coupled to the controller 172. Some non-limiting examples of the grippers 320, 322 include mechanical grippers (such as a parallel-jaw gripper, three-finger gripper, a needle gripper, etc.), electromagnetic grippers, pneumatic grippers, hydraulic grippers, etc. In some embodiments, the grippers 320, 322 may have identical configurations. In other embodiments, the grippers 320, 322 may have differing configurations (e.g., different sizes and/or types). As shown, each of the grippers 320, 322 includes two fingers 330 (distal projections) that are controlled to move in medial and lateral directions along a respective dimension D1, D2 to grip and release objects that are positioned between the fingers 330. The fingers 330 are shown as rigid members, but in other embodiments, the fingers 330 may be articulable.

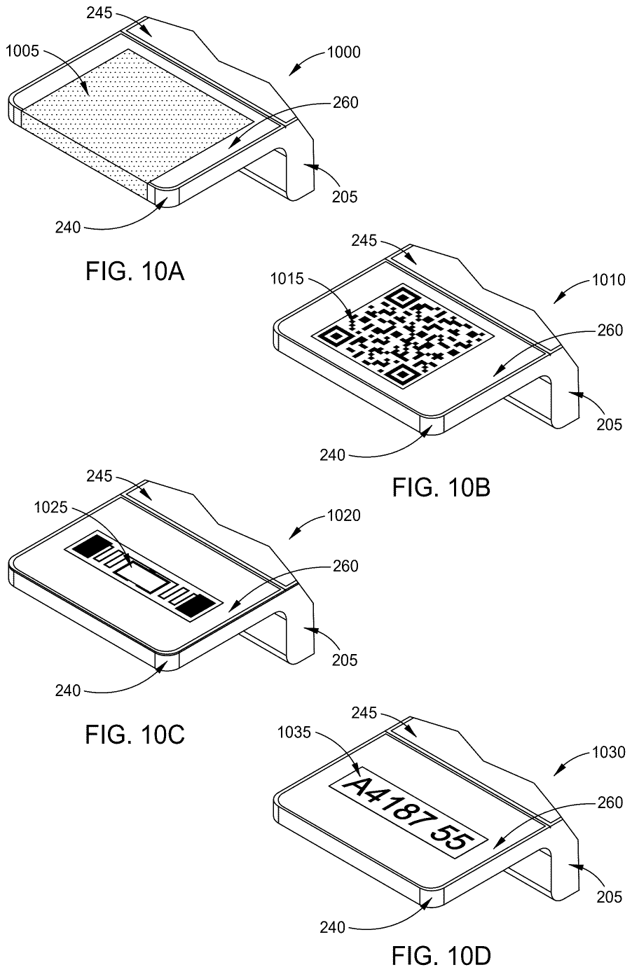

In other embodiments, the end effector arm 310 and the end effector arm 312 may have different types. For example, the end effector arm 310 may include the gripper 320, and the end effector arm 312 may include one or more sensors that are operatively coupled to the controller 172. The sensor data acquired by the one or more sensors may be used by the controller 172 to identify the surgical package 200, to identify its position and orientation, to determine a type of the surgical package 200 (e.g., identifying which items are stored in the surgical package 200), and so forth. The one or more sensors may have any suitable implementation, such as load cells to determine a weight and/or center of gravity of the surgical package 200, an x-ray source and sensor that image the contents of the surgical package 200, a fluorescent light source and camera that illuminate and image a fluorescent identification tag on the surgical package 200, and so forth. Further, the one or more sensors may be configured to sense an identifier included in the projecting interface 260, some examples of which are discussed below with respect to FIGS. 10A-10D.

In some embodiments, the controller 172 may be further configured to control the end effector arm 310 (and the gripper 320) to arrange the surgical package 200 with a desired position and/or orientation relative to the end effector arm 312, which thereby “presents” the surgical package 200 to be sensed by the one or more sensors.

In some embodiments, the instrument library 176 may store additional information associated with the surgical package 200, such as a type of the projecting interface 260, the location and/or orientation of item(s) within the surgical package 200, and so forth. After identifying the item(s) within the surgical package 200, the controller 172 may be further configured to determine (or retrieve) this additional information from the instrument library 176, which may assist in opening the surgical package 200, as described below.

FIG. 4A is a diagram illustrating an exemplary surgical package 400 having a film 410 extending across an opening 405 extending through the body 205, and FIG. 4B is a diagram illustrating exemplary end effector manipulation of the surgical package 400, according to one or more embodiments. The features illustrated in FIGS. 4A, 4B may be used in conjunction with other embodiments. For example, the surgical package 400 represents one example implementation of the surgical package 200 of FIG. 2.

The body 205 defines a second portion 265 (e.g., the tab 240), and the opening 405 extends through the second portion 265, e.g., between an upper surface and a lower surface of the second portion 265. The opening 405 may have any suitable size and shape, as well as positioning relative to the surfaces of the second portion 265. As shown, the opening 405 is circular, is approximately centered on the upper surface and the lower surface, and extends perpendicular thereto.

The film 410 extends across the opening 405. In some embodiments, one or more surfaces of the film 410 are coplanar with the upper surface and/or the lower surface of the second portion 265. In other embodiments, the film 410 may be arranged at a height between the upper surface and the lower surface of the second portion 265. The film 410 may be formed of any suitable material(s) that can be sterilized, e.g., a plastic film such as PE or PET, a metal foil, a paper layer, and so forth. The film 410 may be attached to the second portion 265 using any suitable techniques. For example, the film 410 may be adhered to the upper surface or to the lower surface, or the second portion 265 may be formed around the film 410.

Referring to diagram 425 of FIG. 4B, the end effector arm 312 includes a gripper 430 having a first finger 330 and a second finger 435 with a sharp end 440. The gripping functionality of the gripper 430 may be similar to that of the gripper 320. The sharp end 440 may have any implementation suitable for piercing the film 410, such as a needle or blade. The second finger 435 may have a greater (distal) length than the first finger 330 due to the sharp end 440, but other implementations may have a same length or a lesser length than the first finger 330. The sharp end 440 may be rigidly positioned within the second finger 435 or may be retractable (e.g., telescoping).

In some embodiments, the gripper 430 is positioned by the controller 172 near the lower surface of the second portion 265, and is moved by the controller 172 to cause the second finger 435 (more specifically, the sharp end 440) to pierce the film 410 and extend through the opening 405. In some cases, the film 410 and/or the second portion 265 may include distinctive visual characteristics (e.g., particular color(s) or pattern(s), fiducial marks, etc.) that improve the ability of the end effector arm 312 to align the second finger 435 with the film 410.

In some embodiments, the first finger 330 is disposed near the periphery of the second portion 265 when the second finger 435 extends through the opening 405. The gripper 430 moves the first finger 330 and the second finger 435, when extended through the opening 405, in a medial direction to grip the second portion 265. In other embodiments, the opening 405 is sufficiently large that both the first finger 330 and the second finger 435 may extend through the opening 405. In this case, the gripper 430 moves the first finger 330 and the second finger 435 in a lateral direction to grip the second portion 265.

Other implementations of the gripper 430 are also contemplated. In one non-limiting example, the first finger 330 may be implemented identically to the second finger 435 (e.g., including a pointed end 440), such that either the first finger 330 or the second finger 435 of the gripper 430 may be used to pierce the film 410 and extend through the opening 405. In another non-limiting example, at least the second finger 435 of the gripper 430 is articulable, such the second finger 435 extends through the opening 405 and may curl toward the second portion 265 to itself apply a gripping force to the second portion 265. In this case, the second finger 435 may or may not be used in combination with the first finger 330 to grip the second portion 265.

When the second portion 265 is gripped by the gripper 430, the gripper 320 is controlled by the controller 172 near the upper surface of the second portion 265 to grip the projecting interface 260 and to gradually displace it from the first portion 225 of the body 205 to overcome the sealing force between the cover 245 and the first portion 225, e.g., using a peeling motion.

In some embodiments, the projecting interface 260 of the cover 245 fully or partially overlaps the opening 405 and the film 410. As mentioned above, the projecting interface 260 when in a neutral configuration may contact the upper surface of the second portion 265. In some embodiments, extending the second finger 435 through the opening 405 further operates to displace the projecting interface 260 (e.g., deflecting and/or deforming the projecting interface 260) in a direction away from the upper surface of the second portion 265. Advantageously, reorienting the projecting interface 260 in this manner allows improved access to the projecting interface 260 by the fingers 330 of the gripper 320. Displacing the projecting interface 260 can improve the speed and/or efficiency of opening the surgical package 200, as well as reduce the risk of damage to the body 205 and/or the cover 245 caused by the gripper 320.

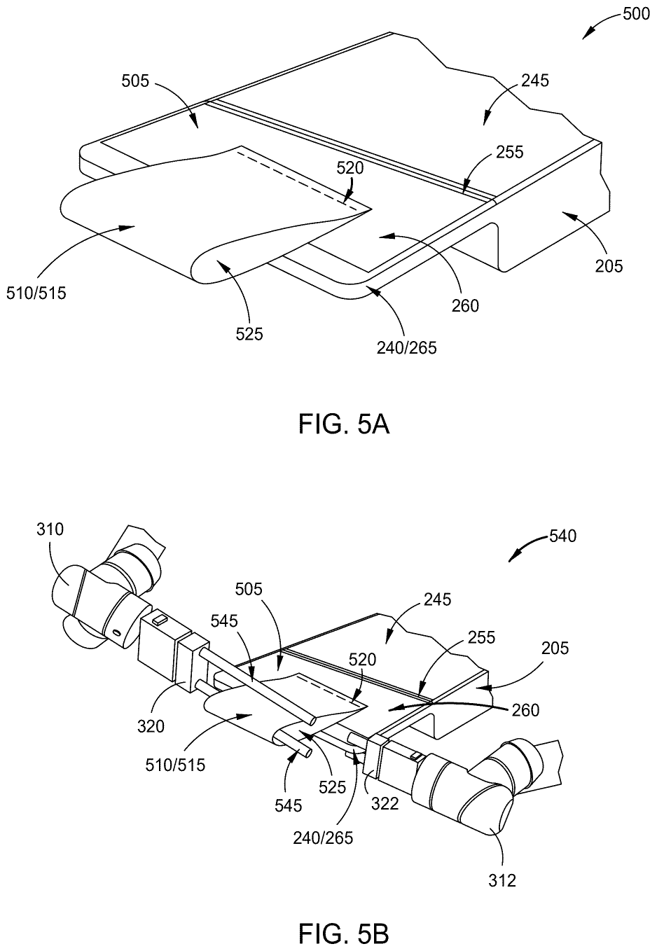

FIG. 5A is a diagram illustrating an exemplary surgical package 500 having a tape loop attached to a surface of a projecting interface, and FIG. 5B is a diagram illustrating exemplary end effector manipulation of the surgical package 500, according to one or more embodiments. The features illustrated in FIGS. 5A, 5B may be used in conjunction with other embodiments. For example, the surgical package 500 represents one example implementation of the surgical package 200 of FIG. 2.

The projecting interface 260 comprises a tape 510, formed as a tape loop 515, that is attached to an upper surface 505 of the projecting interface 260. The tape 510 may be formed of any suitable material(s) that can be sterilized, e.g., a plastic film such as PE or PET. The tape loop 515 overlaps the longitudinal axis of the surgical package 500 and extends along the same dimension. The tape loop 515 defines an aperture 525 that as shown extends beyond an extent of the upper surface 505.

The proximal ends of the tape loop 515 are attached to the upper surface 505 along an axis 520 that is perpendicular to the longitudinal axis, and may be attached using any suitable techniques, such as an adhesive or welding. The tape loop 515 may be attached differently in other implementations, e.g., attached along a parallel axis to the longitudinal axis, attached to an opposing lower surface of the projecting interface 260, and so forth.

Referring to diagram 540 of FIG. 5B, the end effector arm 310 includes a gripper 320 with fingers 545, which in some cases may have a greater (distal) length than the fingers 330. In some embodiments, the gripper 320 is positioned by the controller 172 near the upper surface of the second portion 265, and is moved by the controller 172 to cause one or both of the fingers 545 to extend through the aperture 525. In some cases, the tape loop 515 and/or the second portion 265 may include distinctive visual characteristics (e.g., particular color(s) or pattern(s), fiducial marks, etc.) that improve the ability of the end effector arm 310 to align the finger(s) 545 with the aperture 525.

In some embodiments, one finger 545 extends through the aperture 525 and the other finger 545 is disposed near the periphery of the tape loop 515. The gripper 320 moves the fingers 545 in a medial direction to grip the tape loop 515. In other embodiments, the aperture 525 is sufficiently large that both fingers 545 may extend through the aperture 525. In this case, the gripper 320 moves the fingers 545 in a lateral direction to grip the tape loop 515.

In some embodiments, the gripper 322 of the end effector arm 312 grips a portion of the body 205, such as the second portion 265. When the second portion 265 is gripped, the gripper 320 is controlled by the controller 172 to grip the tape loop 515 and to gradually displace the projecting interface 260 from the first portion 225 of the body 205 to overcome the sealing force between the cover 245 and the first portion 225, e.g., using a peeling motion.

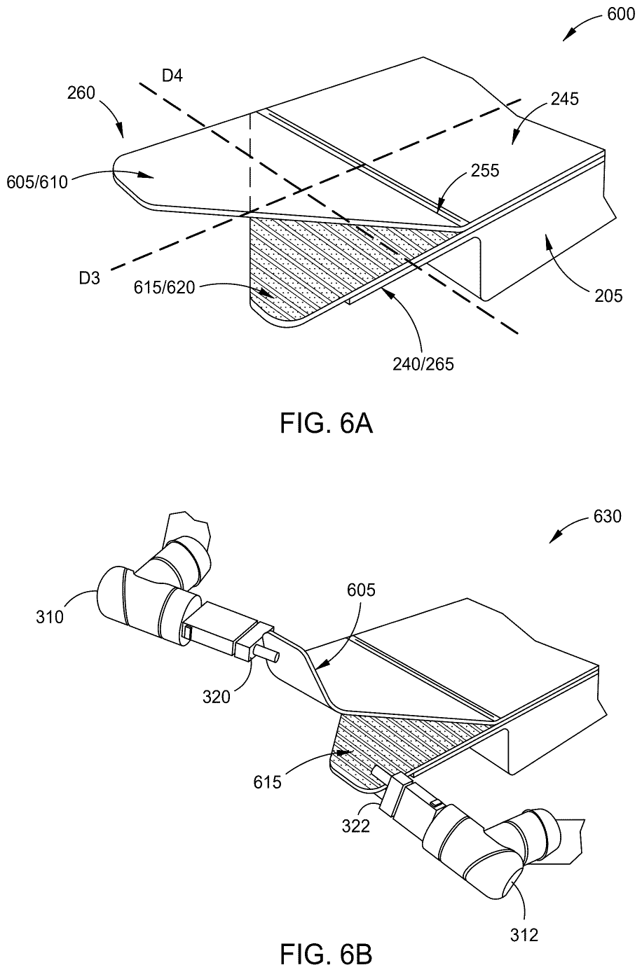

FIG. 6A is a diagram illustrating an exemplary surgical package 600 having opposing tabs for manipulation by an end effector, and FIG. 6B is a diagram illustrating exemplary end effector manipulation of the surgical package 600, according to one or more embodiments. The features illustrated in FIGS. 6A, 6B may be used in conjunction with other embodiments. For example, the surgical package 600 represents one example implementation of the surgical package 200 of FIG. 2.

In the surgical package 600, the projecting interface 260 defines a first tab 605 and a second tab 615 that extend beyond the areal extent of the first portion along a first dimension D3. The first tab 605 and the second tab 615 oppose each other along a second dimension D4, which as shown is perpendicular to the first dimension D3.

The first tab 605 and the second tab 615 may be formed of any suitable materials, and may have the same or a different material composition as each other. In some embodiments, the first tab 605 and the second tab 615 are formed of a same material as the cover 245. In other embodiments, the first tab 605 is formed of a same material as the cover 245, and the second tab 615 is formed of a same material as the body 205 and/or the second portion 265. In yet other embodiments, the first tab 605 and/or the second tab 615 are formed of material(s) different than those of the cover 245 and the body 205.

In some embodiments, one or both of the first tab 605 and the second tab 615 are fully or partially overlapping with the second portion 265, and the first tab 605 and the second tab 615 may be partially overlapping each other. As shown, the first tab 605 is disposed partially over the second tab 615 (e.g., contacting an upper surface 620 of the second tab). In some embodiments, both the first tab 605 and the second tab 615 are movable relative to the second portion 265. In other embodiments, the second tab 615 may be rigidly attached (e.g., adhered or welded) to the second portion 265, or may be integrally formed therewith.

Referring to diagram 630 of FIG. 6B, the gripper 320 of the end effector arm 310 grips the first tab 605, and the gripper 322 of the end effector arm 312 grips the second tab 615 (and in some cases, the second portion 265). In some cases, the first tab 605 and/or the second tab 615 may include distinctive visual characteristics (e.g., particular color(s) or pattern(s), fiducial marks, etc. which may differ from each other) that improve the ability of the end effector arms 310, 312 to arrange the grippers 320, 322 relative to the respective tabs 605, 615.

When the first tab 605 and the second tab 615 are gripped, one or both of the grippers 320, 322 are controlled by the controller 172 to gradually displace the projecting interface 260 from the first portion 225 of the body 205 to overcome the sealing force between the cover 245 and the first portion 225, e.g., using a peeling motion.

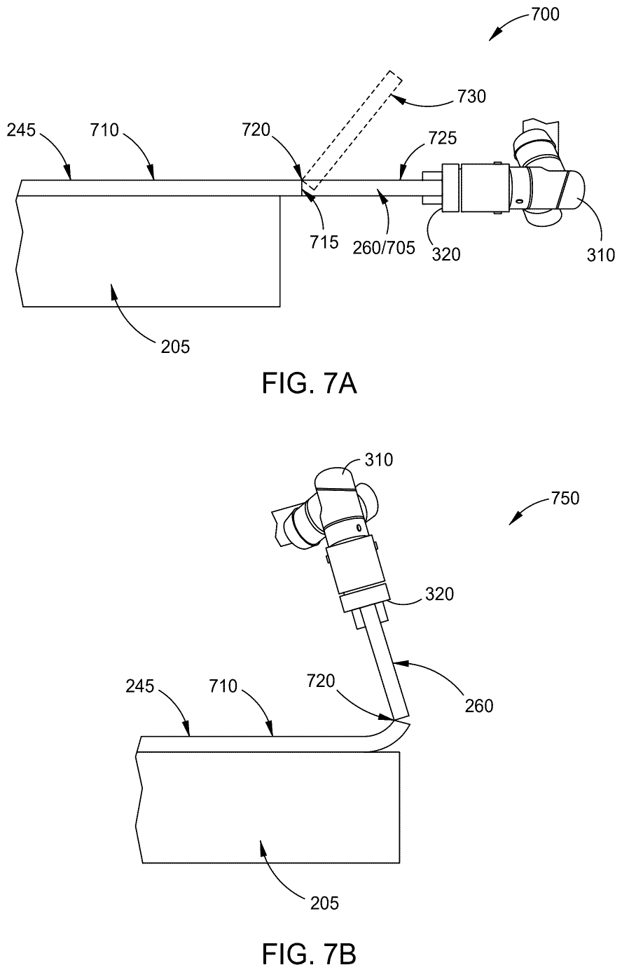

FIGS. 7A, 7B are diagrams illustrating exemplary end effector manipulation of a surgical package 700 with portions of a cover having an attachment force, according to one or more embodiments. The features illustrated in FIGS. 7A, 7B may be used in conjunction with other embodiments. For example, the surgical package 700 represents one example implementation of the surgical package 200 of FIG. 2.

The projecting interface 260 comprises a second portion 705 and the cover 245 comprises a third portion 710. As shown, the third portion 710 covers the first portion 225 and extends beyond an extent of the body 205. When attached together, the second portion 705 extends from the third portion 710 and is generally aligned therewith.

The second portion 705 is initially attached to the third portion 710 along an attachment interface 715. When the surgical package 700 is in the attached configuration, the second portion 705 is in a first position 725. As shown, the second portion 705, the third portion 710, and/or the attachment interface 715 may define one or more planar surfaces of the cover 245 (e.g., upper surface and/or lower surface).

At the attachment interface 715, an attachment force holds the second portion 705 and the third portion 710 together. In some embodiments, an adhesive provides the attachment force. In other embodiments, material of the cover 245 provides the attachment force. For example, the attachment interface 715 may be integrally formed with the second portion 705 and the third portion 710, but contoured differently so as to provide a controlled separation of the second portion 705 from the third portion 710 responsive to force applied by the gripper 320 to the second portion 705. For example, the attachment interface 715 may be formed to be relatively weaker than the second portion 705 and/or the third portion 710, e.g., by including a thinner material, perforations, etc. such that plastic deformation (or partial fracture) of the cover 245 is likely to occur first at the attachment interface 715.

The attachment interface 715 may define a pivot axis 720 extending along a width of the cover 245. In some embodiments, the portion of the attachment interface 715 that defines the pivot axis may be relatively stronger than the other portion(s) of the attachment interface 715 (e.g., omitting the thinner material and/or perforations). The second portion 705 is pivotable relative to the third portion 710 along the pivot axis 720, e.g., pivoting from the first position 725 to a second position 730.

Referring to diagram 750 of FIG. 7B, the gripper 320 of the end effector arm 310 grips the projecting interface 260 (e.g., the second portion 705). The controller 172 controls the gripper 320 to gradually displace the projecting interface 260 from the first portion 225 of the body 205 to overcome the sealing force between the cover 245 and the first portion 225, e.g., using a peeling motion.

According to one exemplary sequence, the gripper 320 overcomes the attachment force of the attachment interface 715, pivoting the second portion 705 from the first position 725 to the second position 730 as in FIG. 7A. The pivot axis 720 portion of the attachment interface 715 is dimensioned such that further movement by the gripper 320 does not cause a complete fracture of the attachment interface 715, but causes the projecting interface 260 to begin separating from the first portion 225 as in FIG. 7B.

In some embodiments, pivoting the second portion 705 may be performed at a different time than the separation of the projecting interface 260. For example, the second portion 705 may be “pre-folded” when storing the surgical package 700, and the projecting interface 260 may be later separated by the gripper 320 when the surgical package 700 is retrieved from storage and opened. Further, pivoting the second portion 705 may be performed by means other than the gripper 320 (e.g., by a technician or by a different robotic system).

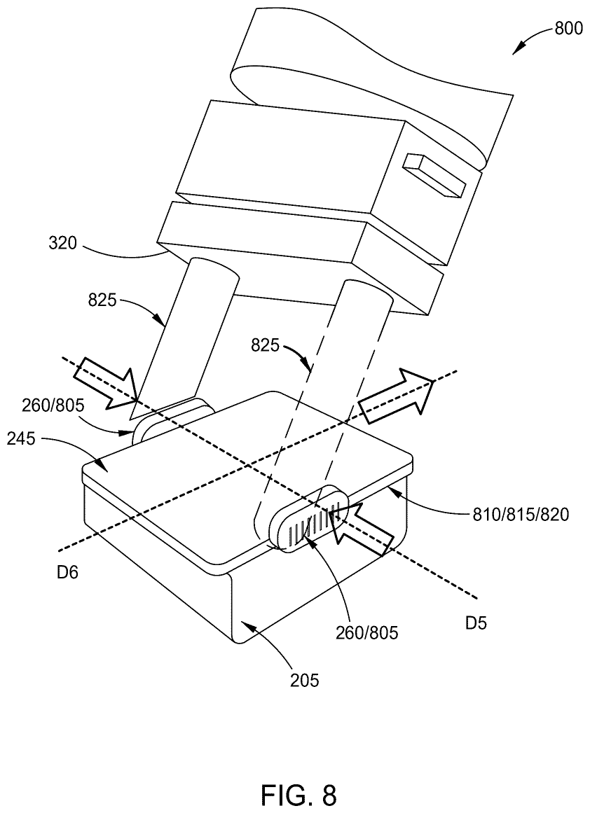

FIG. 8 is a diagram illustrating an exemplary surgical package 800 having a retention mechanism, according to one or more embodiments. The features illustrated in FIG. 8 may be used in conjunction with other embodiments. For example, the surgical package 800 represents one example implementation of the surgical package 200 of FIG. 2.

The surgical package 800 comprises the body 205 and the cover 245. In some embodiments, at least the cover 245 is formed of a rigid material, such as PE, PP, or PET. In some embodiments, some or all of the body 205 is also formed of a rigid material. In one example, the entire body 205 is formed of PE, PP, or PET. In another example, an upper portion of the body 205 is formed of a rigid material and attached to a lower portion of the body 205 that is formed of a flexible material, such as non-woven fabric of PE or PP.

In some embodiments, the cover 245 includes features 810 (e.g., surfaces) near its lateral extents (along a first dimension D5) that are contoured to slidingly engage corresponding features 815 (e.g., channels or rails) formed near the lateral extents of the body 205. In some embodiments, the sealing surface 248 is defined on a lower surface of the cover 245. In some embodiments, the features 810 or features 815 may partially define the sealing surface 248.

The projecting interface 260 comprises pads 805 that are disposed on opposing sides of the cover 245 along the first dimension D5. The pads 805 are operatively coupled to the features 810 of the cover 245. In this way, fingers 825 of the gripper 320 are able to contact the pads 805 and slide the cover 245 relative to the body 205 along a second dimension D6, which as shown is perpendicular to the first dimension D5.

In some embodiments, the surgical package 800 further comprises a retention mechanism 820 that retains the cover 245 to (the first portion 225 of) the body 205. More specifically, the retention mechanism 820 may comprise a detent, latch, etc. that retains the features 810, 815 in a predefined arrangement. When the pads 805 are in a neutral configuration, the retention mechanism 820 retains the cover 245 to the first portion 225. The controller 172 controls the gripper 320 to medially depress the pads 805 (along the first dimension D5) to release the retention mechanism 820, and while the pads 805 are depressed, slide the cover 245 along the second dimension D6 to manipulate the projecting interface 260. This sliding motion is effective to overcome the sealing force between the cover 245 and the first portion 225.

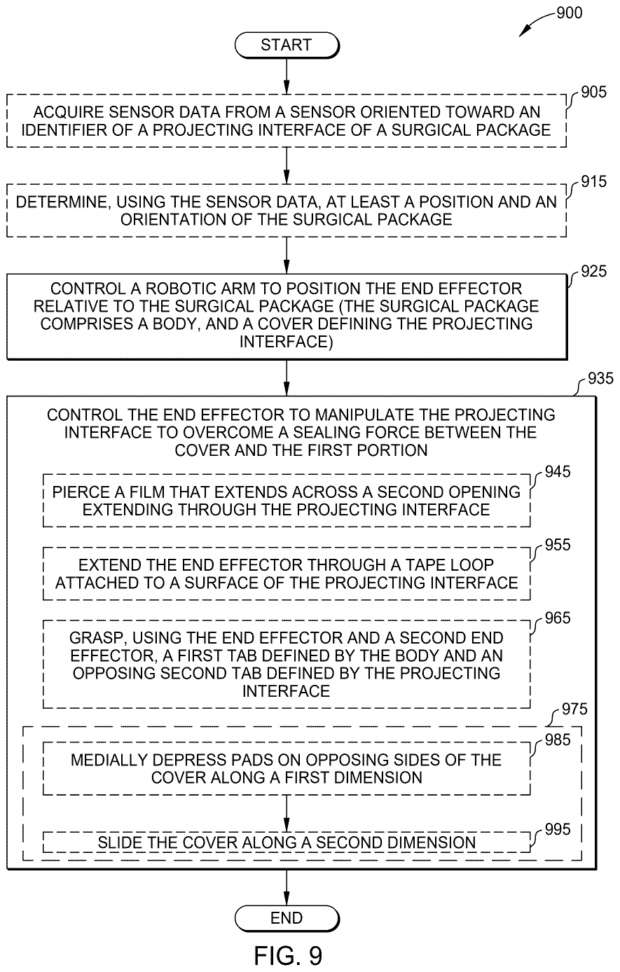

FIG. 9 is an exemplary method 900 of operating an end effector of a robotic system to open a surgical package, according to one or more embodiments. The method 900 may be used in conjunction with other embodiments. For example, the controller 172 may perform the method 900 using end effector(s) 120 such as the grippers 320, 322.

The method 900 begins at optional block 905, where the controller 172 acquires sensor data from a sensor oriented toward an identifier of a projecting interface 260 of a surgical package (such as the surgical packages 200, 400, 500, 600, 700, 800). Some non-limiting examples of identifiers are depicted in FIGS. 10A-10D.

In one example, in diagram 1000 of FIG. 10A, an identifier 1005 is disposed on the upper surface and/or the lower surface of the projecting interface 260. The identifier 1005 comprises one or more visual attributes, such as a color, a pattern, a shape, a fiducial mark, etc. that is identifiable by the controller 172.

In another example, in diagram 1010 of FIG. 10B, an identifier 1015 is disposed on the upper surface of the projecting interface 260. The identifier 1015 comprises a machine-readable code (e.g., a Quick Response (QR) code) that is identifiable by the controller 172.

In another example, in diagram 1020 of FIG. 10C, an identifier 1025 is disposed on or in the projecting interface 260. The identifier 1025 comprises a Radio Frequency Identification (RFID) tag that is operatively coupled to the controller 172.

In another example, in diagram 1030 of FIG. 10D, an identifier 1035 is disposed on the upper surface of the projecting interface 260. The identifier 1025 comprises a machine-readable code, and more specifically, an alphanumeric code that is identifiable by the controller 172 (e.g., using optical character recognition (OCR)).

At optional block 915, the controller 172 determines, using the sensor data, at least a position and an orientation of the surgical package. In some embodiments, the controller 172 further determines which items are stored in the surgical package, e.g., by using the sensor data to access the instrument library 176.

At block 925, the controller 172 controls a robotic arm (e.g., the end effector arms 310, 312) to position the end effector (e.g., the grippers 320, 322) relative to the surgical package. The surgical package comprises a body and a cover that defines the projecting interface 260.

At block 935, the controller 172 controls the end effector to manipulate the projecting interface to overcome a sealing force between the cover and the first portion. In some embodiments, manipulating the projecting interface includes one or more of the following: (at optional block 945) piercing a film that extends across a second opening extending through the projecting interface, (at optional block 955) extending the end effector through a tape loop attached to a surface of the projecting interface, (at optional block 965) grasping, using the end effector and a second end effector, a first tab defined by the body and an opposing second tab defined by the projecting interface, and (within optional block 975) (at optional block 985) medially depressing pads on opposing sides of the cover along a first dimension and (at optional block 995) sliding the cover along a second dimension. The method 900 ends following completion of the block 935.

The present disclosure may be embodied in other specific forms without departing from its spirit or essential characteristics. The described embodiments are to be considered in all respects only as illustrative and not restrictive. The scope of the present disclosure is, therefore, indicated by the appended Claims rather than by this Detailed Description. All changes which come within the meaning and range of equivalency of the Claims are to be embraced within their scope.

Reference throughout this specification to features, advantages, or similar language does not imply that all the features and advantages that may be realized with the present disclosure should be or are in any single embodiment of the disclosure. Rather, language referring to the features and advantages is understood to mean that a specific feature, advantage, or characteristic described in connection with an embodiment is included in at least one embodiment of the present disclosure. Thus, discussions of the features and advantages, and similar language, throughout this specification may, but do not necessarily, refer to the same embodiment.

Furthermore, the described features, advantages, and characteristics of the disclosure may be combined in any suitable manner in one or more embodiments. One skilled in the relevant art will recognize, in light of the description herein, that the disclosure can be practiced without one or more of the specific features or advantages of a particular embodiment. In other instances, additional features and advantages may be recognized in certain embodiments that may not be present in all embodiments of the present disclosure.

Reference throughout this specification to “one embodiment”, “an embodiment”, or similar language means that a particular feature, structure, or characteristic described in connection with the indicated embodiment is included in at least one embodiment of the present disclosure. Thus, the phrases “in one embodiment”, “in an embodiment”, and similar language throughout this specification may, but do not necessarily, all refer to the same embodiment.

The foregoing description is provided to enable any person skilled in the art to practice the various embodiments described herein. Various modifications to these embodiments will be readily apparent to those skilled in the art, and the generic principles defined herein may be applied to other embodiments. Thus, the claims are not intended to be limited to the embodiments shown herein but are to be accorded the full scope consistent with the language of the claims.

### Example Embodiments

Embodiment 1. A system comprises: a robotic arm comprising at least one end effector; and a controller configured to: control the robotic arm to position the end effector relative to a surgical package comprising a body and a cover defining a projecting interface, the projecting interface extending beyond an areal extent of a first portion of the body that defines a first opening into an interior volume of the body; and control the end effector to manipulate the projecting interface to overcome a sealing force between the cover and the first portion.

Embodiment 2. The system of Embodiment 1, further comprising: at least one sensor oriented toward an identifier of the projecting interface, wherein the controller is further configured to: acquire sensor data from the sensor; and determine, using the sensor data, at least a position and an orientation of the surgical package, and wherein positioning the end effector is based on the position and the orientation of the surgical package.

Embodiment 3. The system of Embodiment 1, wherein the body further defines a second portion that defines a second opening extending therethrough, wherein the body further comprises a film extending across the second opening, and wherein manipulating the projecting interface comprises piercing the film.

Embodiment 4. The system of Embodiment 1, wherein the projecting interface comprises: a tape loop attached to a surface of the projecting interface, wherein manipulating the projecting interface comprises extending the end effector through the tape loop.

Embodiment 5. The system of Embodiment 1, wherein the projecting interface defines a first tab and a second tab that extend beyond the areal extent of the first portion along the first dimension, wherein the first tab and the second tab are opposing each other along a second dimension, wherein manipulating the projecting interface comprises: grasping the first tab using the end effector; and grasping the second tab using a second end effector of the robotic arm.

Embodiment 6. The system of Embodiment 1, wherein the projecting interface comprises a second portion and the cover comprises a third portion, and wherein manipulating the projecting interface comprises: overcoming an attachment force between opposing endfaces of the second portion and the third portion, such that the second portion is pivotable relative to the third portion.

Embodiment 7. The system of Embodiment 1, the surgical package further comprising: a retention mechanism, wherein the projecting interface comprises pads on opposing sides of the cover along a first dimension, wherein when the pads are in a neutral configuration, the retention mechanism retains the cover to the first portion, and wherein the end effector is configured to: medially depress the pads to release the retention mechanism; and while the pads are depressed, slide the cover along a second dimension to manipulate the projecting interface.

What is claimed is:

1. A surgical package to facilitate opening by an end effector of a robotic system, the surgical package comprising:
a body defining an interior volume configured to store one or more surgical items therein, wherein a first portion of the body defines a first opening into the interior volume; and
a cover defining:
a sealing surface configured to contact the first portion to cover the first opening and isolate the interior volume from ambient; and
a projecting interface that extends beyond an areal extent of the first portion, the projecting interface configured to be manipulated by the end effector to overcome a sealing force between the cover and the first portion.

2. The surgical package of claim 1,
wherein the body further defines a second portion that defines a second opening extending therethrough,
wherein the body further comprises a film extending across the second opening,
wherein the end effector is configured to pierce the film to manipulate the projecting interface.

3. The surgical package of claim 1, wherein the projecting interface comprises:
a tape loop attached to a surface of the projecting interface,
wherein the end effector is configured to extend through the tape loop to manipulate the projecting interface.

4. The surgical package of claim 1,
wherein the projecting interface defines a first tab and a second tab that extend beyond the areal extent of the first portion along a first dimension,
wherein the first tab and the second tab are opposing each other along a second dimension,
wherein the end effector is configured to grasp the first tab, and
wherein the robotic system further comprises a second end effector configured to grasp the second tab.

5. The surgical package of claim 1,
wherein the projecting interface comprises a second portion and the cover comprises a third portion,
wherein the projecting interface is configured to be manipulated by the end effector to overcome an attachment force provided between opposing endfaces of the second portion and the third portion, such that the second portion is pivotable relative to the third portion.

6. The surgical package of claim 1, further comprising:
a retention mechanism,
wherein the projecting interface comprises pads on opposing sides of the cover along a first dimension,
wherein when the pads are in a neutral configuration, the retention mechanism retains the cover to the first portion, and
wherein the end effector is configured to:
medially depress the pads to release the retention mechanism; and
while the pads are depressed, slide the cover along a second dimension to manipulate the projecting interface.

7. The surgical package of claim 1, wherein the projecting interface includes an identifier that configures the robotic system to determine at least a position and an orientation of the surgical package.

8. The surgical package of claim 7, wherein the identifier comprises one or more visual attributes of a top surface of the projecting interface.

9. The surgical package of claim 7, wherein the identifier comprises a machine-readable code on a top surface of the projecting interface.

10. The surgical package of claim 7, wherein the identifier comprises a Radio Frequency Identification (RFID) tag in the projecting interface.

11. A system comprising:
a robotic arm comprising at least one end effector; and
a controller configured to:
control the robotic arm to position the end effector relative to a surgical package comprising a body and a cover defining a projecting interface, the projecting interface extending beyond an areal extent of a first portion of the body that defines a first opening into an interior volume of the body; and
control the end effector to manipulate the projecting interface to overcome a sealing force between the cover and the first portion.

12. The system of claim 11, further comprising:
at least one sensor oriented toward an identifier of the projecting interface,
wherein the controller is further configured to:
acquire sensor data from the sensor; and
determine, using the sensor data, at least a position and an orientation of the surgical package, and
wherein positioning the end effector is based on the position and the orientation of the surgical package.

13. A method of operating an end effector of a robotic system to open a surgical package, the method comprising:
controlling a robotic arm to position the end effector relative to the surgical package, the surgical package comprising:
a body; and
a cover defining a projecting interface, the projecting interface extending beyond an areal extent of a first portion of the body that defines a first opening into an interior volume of the body; and
controlling the end effector to manipulate the projecting interface to overcome a sealing force between the cover and the first portion.

14. The method of claim 13, further comprising:
acquiring sensor data from a sensor oriented toward an identifier of the projecting interface; and
determining, using the sensor data, at least a position and an orientation of the surgical package,
wherein positioning the end effector is based on the position and the orientation of the surgical package.

15. The method of claim 13, wherein manipulating the projecting interface comprises one of:
piercing a film that extends across a second opening extending through a second portion of the body;
extending the end effector through a tape loop attached to a surface of the projecting interface;
grasping, using the end effector and a second end effector, opposing first and second tabs defined by the projecting interface; and
medially depressing pads on opposing sides of the cover along a first dimension, and sliding the cover along a second dimension.
